(19) United States
(12) Patent Application Publication
(10) Pub. No.: US 2021/0128845 A1
(43) Pub. Date: May 6, 2021

(54) DEVICE AND METHOD FOR INJECTING MEDICINE

(71) Applicant: PHILOSYS CO., LTD., Gunsan-si (KR)

(72) Inventors: Woo Hyek Choi, Yongin-si (KR); Jeong Sik Kim, Seoul (KR); Eun Su Cho, Yongin-si (KR); Young Min Park, Seoul (KR)

(21) Appl. No.: 17/030,327

(22) Filed: Sep. 23, 2020

(30) Foreign Application Priority Data
Oct. 30, 2019 (KR) 10-2019-136219

(51) Int. Cl. A61M 5/50; A61M 5/24

(52) U.S. Cl. CPC A61M 5/5086; A61M 5/24; A61M 2205/3368; A61M 2205/18; A61M 2205/582; A61M 2205/587; A61M 2205/583; A61M 2205/581; A61M 2205/3584

(57) ABSTRACT

According to an embodiment, a medicine injection device comprises a medicine storage unit receiving a medicine cartridge storing a medicine, a needle unit connected with the medicine cartridge and injecting the medicine to a user, a driver configured to apply an external force to the medicine storage unit, a temperature sensor measuring a temperature of at least one of the medicine storage unit, the needle unit, the driver, and an outside of the medicine injection device, and a controller determining a medicine state indicating whether the medicine deteriorates based on the temperature measured by the temperature sensor and controlling the driver according to the determined medicine state to thereby determine whether to block the injection of the medicine.

### CROSS-REFERENCE TO RELATED APPLICATIONS

This application claims priority to Korean Patent Application No. 10-2019-136219 filed in the Korean Intellectual Property Office on Oct. 30, 2019, the disclosure of which is incorporated by reference herein in its entirety.

### TECHNICAL FIELD

The disclosure relates to technology for injecting a medicine based on the temperature sensed by a device.

### DISCUSSION OF RELATED ART

In general, medicine needs to be stored at an appropriate temperature.

When stored at an improper storage temperature, medicine may go bad and fail to work properly, even causing side effects.

Thus, for portable medicine injection devices, the ambient temperature or device temperature may be critical.

For example, insulin needs to be stored in a refrigerator if not opened and, once opened, should be stored at an appropriate temperature (room temperature or 1° C. to 30° C.).

Patients with diabetes tend to carry an insulin injection device all the time for injection of insulin from time to time. In the summer or winter, insulin may be easily deteriorated according to the ambient temperature.

The deteriorated insulin does not work properly, resulting in a failure to adjust blood sugar and hence putting the patient in critical condition.

Medicine deterioration is not easily identified with the naked eye. Thus, medicine needs to be separately managed if exposed long to a temperature higher than the proper temperature.

### SUMMARY

According to an embodiment, the medicine injection device may prevent a patient from using medicine which has been stored at a temperature out of the appropriate storage temperature.

According to an embodiment, a medicine injection device comprises a medicine storage unit receiving a medicine cartridge storing a medicine, a needle unit connected with the medicine cartridge and injecting the medicine to a user, a driver configured to apply an external force to the medicine storage unit, a temperature sensor measuring a temperature of at least one of the medicine storage unit, the needle unit, the driver, and an outside of the medicine injection device, and a controller determining a medicine state indicating whether the medicine deteriorates based on the temperature measured by the temperature sensor and controlling the driver according to the determined medicine state to thereby determine whether to block the injection of the medicine.

When the temperature measured by the temperature sensor is less than a second threshold temperature and not less than a first threshold temperature, the controller may determine that the medicine state is a danger state in which the medicine has a chance of deterioration.

When the temperature measured by the temperature sensor is not less than the second threshold temperature, the controller may determine that the medicine state is a deteriorated state in which the medicine deteriorates.

The medicine injection device may further comprise an alarm unit outputting an alarm indicating the medicine state determined by the controller. When the controller determines that the medicine is in a danger state, the alarm unit may output a danger alarm and, when the controller determines that the medicine is in a deterioration state, the alarm unit may output a deterioration alarm.

Upon determining that the medicine state is a deterioration state, the controller may stop the operation of the driver to block the injection of the medicine.

In response to a controlling of the medicine injection by the controller, the driver may move a piston of the medicine cartridge received in the medicine storage unit from a rear end of the medicine injection device to a front end of the medicine injection device.

Upon determining that medicine state is a deteriorated state, the controller may control the driver to block the injection of the medicine and, in response to a return, to an initial position, of the piston, allow the injection of the medicine.

After the medicine injection is blocked, the driver may move the piston from the front end of the medicine injection device to the initial position of the rear end.

Upon determining that the medicine state is the deteriorated state, the controller may lock an advance of the piston by the driver, track the position of the piston after the driver is locked, and in response to a determination that the tracked position is the initial position, set the locked driver to an operable state.

In response to detecting a removal of the medicine cartridge from the medicine storage unit after the medicine injection is blocked, the controller may set the driver to an operable state.

When temperatures are measured in a plurality of positions of the medicine storage unit, the needle unit, the driver, and the outside of the medicine injection device, the controller may determine the medicine state based on a highest temperature of the measured temperatures.

When temperatures are measured in a plurality of positions of the medicine storage unit, the needle unit, the driver, and the outside of the medicine injection device, the controller may determine the medicine state based on an average temperature of the measured temperatures.

The medicine injection device may further comprise a communication unit communicating with a user terminal. A result of the measurement may be transferred to the user terminal via the communication unit. A measurement history associated with the measurement result may be stored in the user terminal.

The communication unit may receive a signal indicating the medicine state determined by the user terminal. The controller may control the driver based on the signal to thereby determine whether to block the medicine injection.

In at least one of a case where the medicine is determined to deteriorate by the user terminal or a case where the user's stop control input is detected, a stop control signal may be received from the user terminal.

According to an embodiment, the medicine injection device may detect a chance of deterioration of medicine.

According to an embodiment, the medicine injection device may prevent medicine from fully deterioration by providing a notification to the user before the medicine deteriorates.

According to an embodiment, the medicine injection device may control the operation of the medicine injection device when the medicine in the cartridge deteriorates, thereby preventing side effects due to the deterioration of medicine by blocking the medicine injection.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a perspective view illustrating a medicine injection device according to an embodiment;

FIG. 2 is a block diagram illustrating a configuration of a medicine injection device according to an embodiment;

FIG. 3 is a block diagram illustrating a configuration of a medicine injection device according to an embodiment;

FIG. 4 is a flowchart illustrating the step of determining the state of medicine according to an embodiment;

FIG. 5 is a block diagram illustrating a configuration of a medicine injection device to describe the operation of a driver, according to an embodiment;

FIG. 6 is a flowchart illustrating the step of controlling the operation of a driver while tracking the position of a piston according to an embodiment; and

FIG. 7 is a view illustrating an example of communication with a user terminal according to an embodiment.

### DETAILED DESCRIPTION

The specific structures or functions herein are provided merely for illustration purposes, and various changes or modifications may be made thereto.

Thus, embodiments of the disclosure are not limited to those described herein, and the scope of the disclosure encompasses all modifications, changes, replacements, or equivalents thereof without departing from the technical spirit of the disclosure.

The terms “first” and “second” as used herein may be used to describe various components, simply for distinguishing one element from another, but the components should not be limited by the terms.

For example, a first component may be denoted a second component, and vice versa without departing from the scope of the present disclosure.

When a component is “connected to” or “coupled to” another component, the component may be directly connected or coupled to the other component, or other component(s) may intervene therebetween.

As used herein, the singular forms “a,” “an,” and “the” are intended to include the plural forms as well, unless the context clearly indicates otherwise.

It will be further understood that the terms “comprise” and/or “have,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms including technical and scientific terms used herein have the same meaning as commonly understood by one of ordinary skill in the art to which the embodiments of the present disclosure belong.

It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, embodiments of the disclosure are described below in detail with reference to the accompanying drawings.

The same reference denotations are used to refer to the same members throughout the drawings.

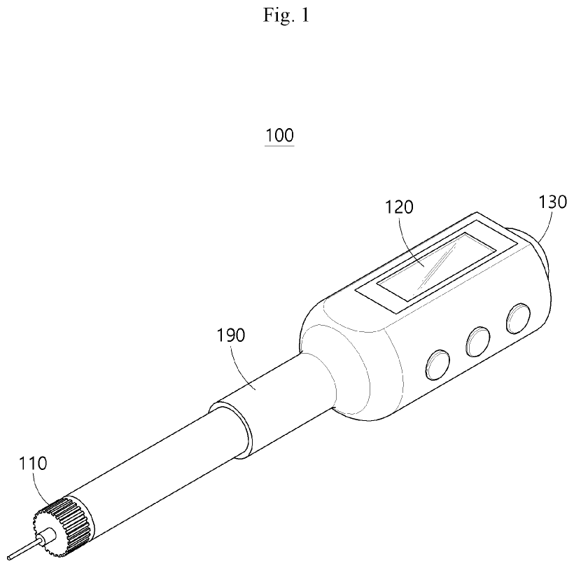

FIG. 1 is a perspective view illustrating a medicine injection device according to an embodiment.

According to an embodiment, a medicine injection device 100 may inject a medicine via invasion.

The amount of medicine injected may be determined based on the amount of a target biological material.

The medicine injection device 100 may receive the amount of the target biological material sensed by an external blood sugar measuring device, but without limitations thereto, the medicine injection device 100 may sense the amount of the target biological material on its own.

As used herein, the term ‘biological material’ may refer to a material associated with a living body.

The biological material may be, or be denoted, an analyte.

For example, the biological material may be blood sugar, but not limited thereto.

The target biological material (target analyte) is blood sugar, and the medicine corresponding thereto may be insulin.

One or more temperature sensors may be provided in at least one position of the outside or inside of the medicine injection device 100, and the medicine injection device 100 may measure the temperature of the medicine injection device 100 via the one or more temperature sensors.

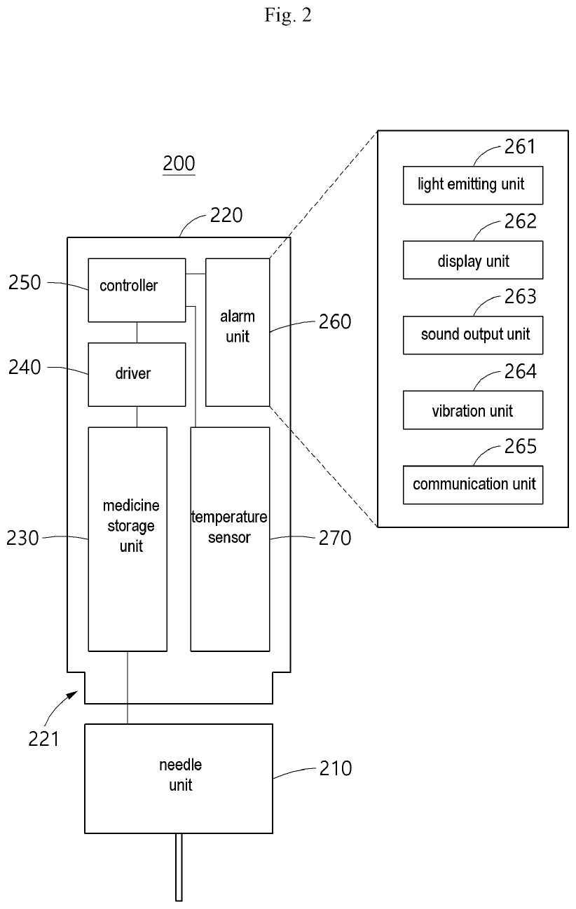

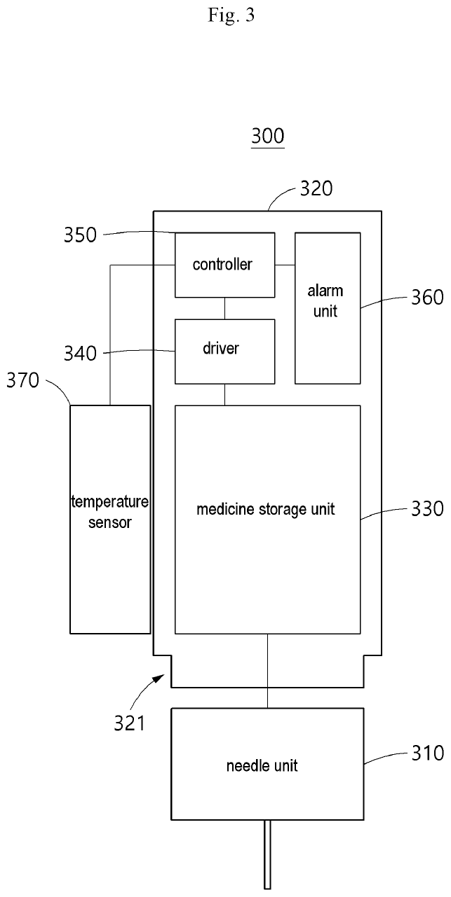

A configuration for measuring the temperature is described below with reference to FIGS. 2 and 3.

The medicine injection device 100 may include a housing 190.

The housing 190 may support a needle unit 110 and a cartridge.

The housing 190 may include an output interface 120 for providing various pieces of information and an input interface 130 for receiving user control or manipulation from the user.

The input interface 130 may include buttons or keys on the side surface of the housing 190.

The output interface 120 may include a display for providing visual information and a speaker for providing sound information.

The input interface 130 may include buttons operated by the user's control action.

The output interface 120 and input interface 130 of FIG. 1 are merely an example, and embodiments of the disclosure are not limited thereto.

The output interface 120 may include an alarm unit 260 shown in FIG. 2.

FIG. 2 is a block diagram illustrating a configuration of a medicine injection device according to an embodiment.

A header 221 of a housing 220 may connect to a needle unit 210.

The header 221 may include a first coupler that may couple with the needle unit 210.

The medicine injection device 200 may connect to the needle unit 210 via the first coupler.

The first coupler may be a structure that may couple with a second coupler of the needle unit 210. The first coupler may be implemented as a coupling structure, such as a screw-type structure or a magnet-type structure.

For example, one of the first coupler and the second coupler may include a thread, and the other thereof may include a thread groove, so that the thread may be engaged with the thread groove.

The housing 220 may be a member that supports a medicine storage unit 230, a driver 240, a controller 250, and an alarm unit 260.

The housing 220 may be implemented as a pen-shaped structure whose length is larger than its width or as a small box-shaped structure the length-width difference of which is smaller than a threshold length.

The shape of the housing 220 may be varied depending on, e.g., the shape and arrangement of the medicine storage unit 230, driver 240, controller 250, and alarm unit 260 embedded therein.

The medicine storage unit 230 may be a member for storing medicine and may receive a cartridge for storing medicine.

When the needle unit 210 and the header 221 of the medicine injection device 200 are coupled together, the medicine storage unit 230 may connect to the needle unit 210, forming a medicine transfer path.

For example, a front end of the needle unit 210 may be exposed from the coupler of the needle unit 210 and extend. A rear end of the needle unit 210 may pass through the header 221 of the housing 220 and directly or indirectly connect to the medicine storage unit 230.

The medicine storage unit 230 may transfer medicine to the needle unit 210.

The cartridge containing medicine may be removed from the medicine storage unit 230.

Thus, the user may mount a cartridge, according to the doctor (physician)'s proscription, in the medicine injection device 200.

The driver 240 may be configured to apply an external force to the medicine storage unit 230.

For example, the driver 240 may apply an external force to the rear end of the medicine storage unit 230, thereby discharging the medicine through a fluid path formed from the front end of the medicine storage unit 230 to the needle unit 210.

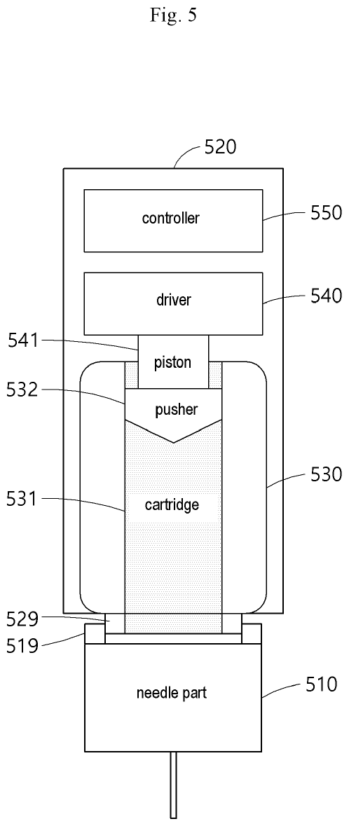

The driver 240 is described below in detail with reference to FIG. 5.

The controller 250 may determine the medicine state, which indicates whether the medicine deteriorates, based on the temperature measured by a temperature sensor.

For example, when the measured temperature is not less than a threshold temperature, the controller 250 may determine whether the medicine has deteriorated.

The controller 250 may control the driver 240 according to the determined medicine state, determining whether to stop (or block) the injection of the medicine.

Upon determining that the medicine is in normal state, the controller 250 may control the driver 240 to operate, thereby allowing the medicine to be injected to the user.

Upon determining that the medicine is in deteriorated state, the controller 250 may lock the driver 240 to block the injection of medicine to the user.

Accordingly, the medicine injection device 200 may block the injection of medicine which is highly likely to deteriorate, preventing side effects due to deteriorated medicine.

With the medicine injection blocked, the controller 250 may perform the other functions or operations except for medicine injection.

With the medicine injection blocked, the controller 250 may disregard a medicine injection command received via the input interface.

The alarm unit 260 may output an alarm or notification indicating the medicine state determined by the controller 250.

The alarm unit 260 may provide the medicine state and the measured temperature information to the user via various means.

For example, the alarm unit 260 may include a light emitting unit 261, a display unit 262, a sound output unit 263, a vibration unit 264, and a communication unit 265.

The light emitting unit 261 may include a light emitting diode (LED). The light emitting unit 261 may output the content or information indicating the medicine state and temperature information via the wavelength or color of the light emitted from the LED, or the symbol or blinking type (e.g., blinking count or duration) indicated by the LED.

For example, when the medicine state is the deteriorated state, the light emitting unit 261 may emit a wavelength or color of light distinguished from the color of the housing to alert the user.

The wavelength or color of light distinguished from the color of the housing may be, e.g., a wavelength of red light.

The display unit 262 may visualize text information or symbol information in graphical representations to indicate, guide, or direct the medicine state and measurement information.

The sound output unit 263 may output a beep or voice to indicate the medicine state and measurement temperature.

The vibration unit 264 may output vibration in a different number of times or vibration duration depending on the medicine state and transfer information related to the medicine state to the user.

The communication unit 265 may communicate with a user terminal and may transmit a signal to the user terminal so that the user terminal may provide an alert, alarm, or notification to the user.

The type of the alarm provided to the user by the user terminal and the medicine injection device 200 is not limited to a specific one, but may rather any type of alarm, alert, or notification may be used to alert or notify the user of the medicine state.

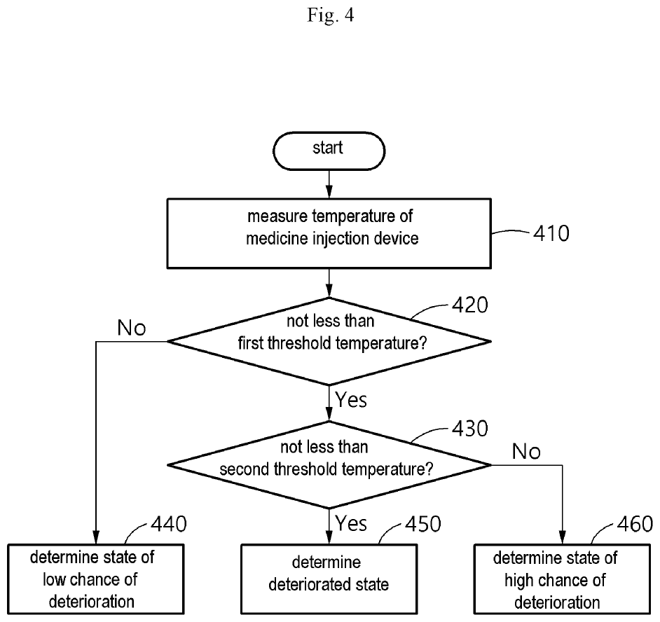

The alarm may be divided into two or more warning levels (e.g., a first warning and a second warning), and an example of providing an alarm per warning level is described below with reference to FIG. 4.

According to an embodiment, when the temperature of the medicine falls out of the appropriate storage temperature range, the alarm unit 260 may display an alert on the display unit 262 of the medicine injection device or may output an LED alert via the light emitting unit 261 disposed around the input interface.

To identify the state, the light emitting unit 261 may be disposed around the input interface of the medicine injection device, and the medicine injection device may provide an alarm regarding the medicine state to the user via the light emitting unit 261.

Accordingly, the medicine injection device 200 may provide the user with the medicine state which may be difficult for the user to determine with the naked eye.

The needle unit 210 may be configured to insert the skin and discharge the medicine.

For example, the needle unit 210 may connect to the medicine injection device 200 and may include a penetration member.

An end (e.g., the front end) of the penetration member may be formed to be able to penetrate the skin, and a path through which the fluid (e.g., the medicine) may flow or move along the lengthwise direction of the penetration member, may be formed inside the penetration member.

The coupler may support a suction penetration member and an injection penetration member and may couple to the housing of the medicine injection device via a structure detachable from the medicine injection device 200.

The temperature sensor 270 may measure the temperature of the inside of the medicine injection device.

For example, the temperature sensor 270 may measure the temperature of at least one of the medicine storage unit 230, needle unit 210, and driver 240 inside the housing 220, but not limited thereto.

The temperature sensor 270 may be a contact-type sensor or a contactless sensor.

When the temperature sensor 270 is a contact-type sensor, the temperature sensor 270 may be disposed in a position where the measurement point of the temperature sensor 270 directly contacts the target object for temperature measurement (e.g., the outer surface of the cartridge).

When the temperature sensor 270 is a contactless sensor, the temperature sensor 270 may measure the temperature using thermal energy radiated from the target object for temperature measurement.

FIG. 3 is a block diagram illustrating a configuration of a medicine injection device 300 according to an embodiment.

The needle unit 310, the medicine storage unit 330, the driver 340, and alarm unit 360 of FIG. 3 are substantially the same as the needle unit 210, medicine storage unit 230, driver 240, and the alarm unit 260, respectively, of FIG. 2, and no further detailed description thereof is given below.

According to an embodiment, the temperature sensor 370 of the medicine injection device 300 may be provided outside of the housing 320 of the medicine injection device 300.

The temperature sensor 370 outside of the housing 320 may be positioned adjacent to the medicine injection device 300, and the temperature sensor 370 may sense or detect the external temperature and the temperature resultant from the driving of the medicine injection device 300.

Although the temperature sensor 270 of FIG. 2 is placed inside the medicine injection device, and the temperature sensor 370 of FIG. 3 is placed outside the medicine injection device, embodiments of the disclosure are not limited thereto.

For example, temperature sensors 370 may be provided in a plurality of positions, e.g., inside and outside of the medicine injection device.

The driver 340 may include a motor for moving the piston of the cartridge received in the medicine storage unit from the rear end to the front end.

Where the temperature sensor 370 is positioned adjacent to the driver 340, the temperature measurement by the temperature sensor 370 may be distorted due to the heat generated as the motor is driven.

Thus, the temperature sensor 370 may be spaced apart from the driver 340 and may be disposed in a position, which is a predetermined distance away from the driver 340.

As the distance from the driver 340 increases, the influence, to the temperature sensor 370, by the heat generated from the driver 340 may reduce.

According to an embodiment, the controller 350 may predict the temperature of the medicine using the temperature measured by the temperature sensor 370.

The controller 350 may determine the temperature of the medicine using at least one of the temperature of the outside of the device and the temperature of the device.

For example, the temperature sensor 370 may measure the temperature in a plurality of positions inside or outside the medicine injection device 300 (e.g., a plurality of positions on the outer surface of the device and/or a plurality of positions inside the device), and the controller 350 may determine the medicine state based on the highest one of the temperatures measured in the plurality of positions.

According to an embodiment, the controller 350 may determine the medicine state based on the average temperature of the temperatures measured in the plurality of positions.

Use of the result of measurement in a plurality of positions, as compared with the result of measurement in a single position, may allow for prediction of the temperature of the medicine within a smaller error range.

FIG. 4 is a flowchart illustrating the step of determining the state of medicine according to an embodiment.

In step 410, the temperature sensor may measure the temperature of the medicine injection device.

In step 420, the controller may determine whether the temperature of the medicine injection device, measured, is a first threshold temperature or more.

The first threshold temperature may be set to, e.g., 30° C.

When the measured temperature is less than the first threshold temperature, the controller may perform step 440, determining that the medicine state is a state in which the medicine is less likely to deteriorate (e.g., normal state).

Upon determining that the temperature of the medicine injection device is the first threshold temperature or more, the controller may perform step 430.

In step 430, the controller may determine whether the measured temperature is a second threshold temperature or more.

The second threshold temperature may be higher than the first threshold temperature.

The second threshold temperature may be set to, e.g., 37° C.

When the measured temperature is less than the second threshold temperature, the controller may perform step 460, determining that the medicine state is a state in which the medicine is highly likely to deteriorate.

When the measured temperature is the second threshold temperature or more, the controller may perform step 450, determining that the medicine is in the deteriorated state.

For example, if the medicine is stored for a long time in the state not less than the first threshold temperature and less than the second threshold temperature, the medicine may deteriorate and thus may not work properly.

For example, when stored at a temperature not more than 30° C., insulin is less likely to deteriorate and to work properly. However, when stored for a predetermined time (e.g., 28 days or longer) within a temperature range from 32° C. to 37° C., the effects of insulin may be reduced by about 14% to about 18%.

If the storage temperature is 37° C. or more, insulin may deteriorate.

When the controller determines that the medicine is in the normal state where the medicine is less likely to deteriorate in step 440, the controller may provide no separate alarm, and the controller may control the driver to inject the medicine to the user.

According to an embodiment, the light emitting unit may emit a first color of light (e.g., light of a wavelength of green light), notifying the user that the medicine is in the normal state.

When the controller determines that the medicine is in the deteriorated state in step 450, the controller may provide an alarm of deterioration (also referred to as a deterioration alarm), and the controller may control the driver to block the injection of the medicine.

The alarm of deterioration may be an alarm corresponding to the second warning level and may be an alarm for providing the user with the measured temperature, medicine state, and medicine injection block message via the sound output unit.

According to an embodiment, the light emitting unit may emit a third color of light (e.g., light of a wavelength of red light) to the user to notify the user that the medicine is in the deteriorated state.

When the controller determines that the medicine is in the state of a high chance of deterioration in step 460, the controller may provide a danger alarm.

The danger alarm may be an alarm corresponding to the first warning level and may be an alarm for providing the user with the measured temperature, medicine state, and a message to indicate the possibility of blocking the injection of medicine via the sound output unit.

According to an embodiment, the light emitting unit may emit a second color of light (e.g., light of a wavelength of yellow light) to the user to notify the user that the medicine is in the danger state.

The alarm unit may provide the user with the danger alarm to lead to a variation in the storage temperature of the medicine injection device, thereby preventing the medicine from fully deteriorating.

According to an embodiment, when the danger state of the medicine lasts for a predetermined period, the controller may control the driver to block the injection of the medicine.

Where the medicine state is the danger state, the chance of immediate deterioration of the medicine is low but, if this state lasts for a long time, the medicine state may be the state of a high chance of deterioration.

Accordingly, the controller may count the time during which the medicine state remains the danger state and, if the counted time is a predetermined period of time or longer, the controller may block the injection of the medicine.

While the medicine injection device is inactive (e.g., turned off), the controller may operate in a sleep mode.

In the sleep mode, the controller may monitor the temperature of medicine by measuring the temperature at every predetermined cycle via the temperature sensor.

The controller may map the measured temperature with the measurement time and store the history.

When at least one of the temperatures measured in the sleep mode is a threshold temperature, the controller may provide at least one of the danger alarm and the deterioration alarm.

For example, at the timing when the temperature measured in the sleep mode exceeds the first threshold temperature, the controller may activate (e.g., turn on) the medicine injection device and provide the danger alarm to the user.

As another example, when there is a timing when the temperature measured in the sleep mode exceeds the first threshold temperature, the controller may provide the danger alarm to the user when the medicine injection device is activated.

Further, at the timing when the temperature measured in the sleep mode exceeds the second threshold temperature, the controller may activate the medicine injection device and provide the deterioration alarm and lock the driver.

When there is a timing when the temperature measured in the sleep mode exceeds the second threshold temperature, the controller may provide the deterioration alarm to the user when the medicine injection device is activated.

Although the danger alarm corresponding to the first warning level and the deterioration alarm corresponding to the second warning level are provided by the medicine injection device in FIG. 4, embodiments of the disclosure are not limited thereto.

For example, at least one of the danger alarm and the deterioration alarm may be provided by the user terminal associated, interlocked or interworking with the medicine injection device.

For example, the medicine injection device may provide the danger alarm as a first warning via the user terminal, and the may include may directly provide the deterioration alarm via a second warning.

Alternatively, the user terminal may provide both the danger alarm and the deterioration alarm.

The medicine injection device may restrict the injection of medicine while providing the deterioration alarm.

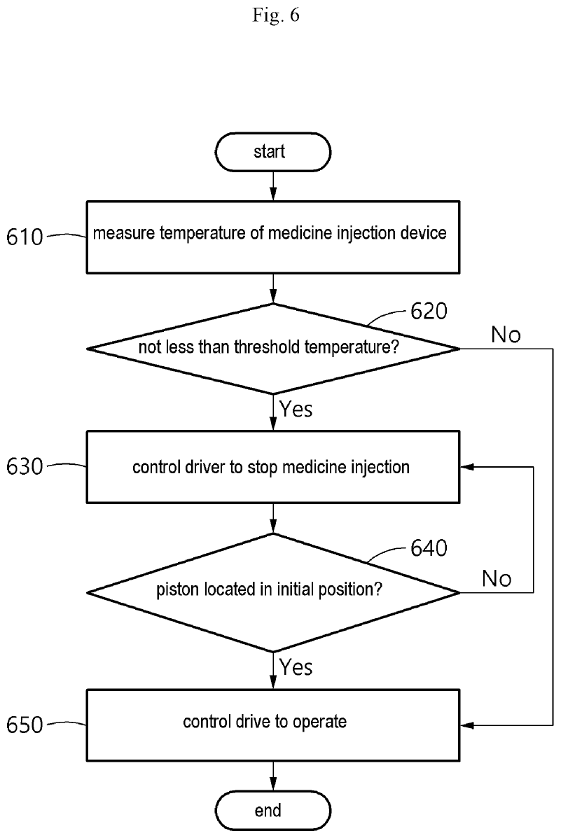

The restriction on the medicine injection is described below with reference to FIGS. 5 and 6.

FIG. 5 is a block diagram illustrating a configuration of a medicine injection device to describe the operation of a driver, according to an embodiment.

A driver 540 may move a piston 541 of a cartridge 531 received in a medicine storage unit 530 from the rear end of the medicine injection device to the front end, in response to medicine injection control by the controller 550.

For example, the driver 540 may include a motor and a linear gear.

The motor may rotate the shaft by the amount of rotation determined by the controller 550.

The linear gear may be engaged with a gear box and connected with the shaft of the motor.

When the motor rotates the shaft, the linear gear may move from the rear end of the medicine injection device to the front end along the lengthwise axis of the medicine injection device.

The controller 550 may move forward the linear gear in response to an injection signal (e.g., a signal according to a command to inject the medicine) to inject the medicine being generated by the user.

The front end of the linear gear may couple to the rear end of the piston 541, and the front end of the piston 541 may connect to the rear end of a pusher 532.

Accordingly, as the controller 550 adjusts the rotation of the motor, the pusher 532 may control the distance in which the pusher 532 advances.

The controller 550 may move forward the piston 541 and the pusher 532 via the driver 540, thereby injecting the medicine via the needle unit 510 connected with the medicine injection device via the first coupler 529 of the housing 520 and the second coupler 519 of the needle unit 510.

However, the driver 540 is not limited to the above-described mechanical structure, and may be implemented as a structure including a pressurizing unit (e.g., an elastic member, such as rubber or spring) pressurizing the rear end of the piston 541 and an injection adjuster for adjusting the amount of medicine injected at the front end of the medicine storage unit 530.

Other various structures capable of injecting the medicine of the cartridge may be used.

FIG. 6 is a flowchart illustrating the step of controlling the operation of a driver while tracking the position of a piston according to an embodiment.

In step 610, the temperature sensor may measure the temperature of the medicine injection device.

In step 620, the controller may determine whether the measured temperature is a threshold temperature or more.

The threshold temperature may be a reference temperature for determining whether the medicine deteriorates and, when the measured temperature is the threshold temperature or more, the controller may determine that the medicine state is the deteriorated state.

In step 630, the medicine injection device may control the driver to stop the medicine injection.

According to an embodiment, upon determining that the medicine state is the deteriorated state, if the measured temperature is the threshold temperature or more, the controller may lock the driver to thereby block the medicine injection.

For example, the controller may lock the advancing of the driver.

In other words, the driver may be set to a state where the driver may move back.

The controller may couple a mechanical structure (e.g., a stopping jaw, or a stopper) for stopping the piston from advancing to the piston and/or the driver or may block the application, to the driver, of the electrical signal indicating the advancing of the piston.

In step 640, the medicine injection device may determine whether the piston is located in the initial position.

According to an embodiment, after the driver is locked, the controller may track the position of the piston and determine whether the tracked position is the initial position of the piston.

The controller may set the driver to the advance-locked state while providing the second warning and then rewind the piston.

To replace the existing cartridge in the medicine storage unit with a new cartridge, the piston may need to be back to the initial position.

For example, in response to the case where the medicine injection is blocked, the controller may move the piston from the front end of the medicine injection device to the initial position of the rear end thereof.

As another example, the controller may move the piston to the initial position in response to a user input.

As another example, the piston may be manually moved by the user.

Where the tracked position of the piston is not the initial position, the controller may maintain the medicine injection-blocked state and keep on tracking the position of the piston.

In step 650, the medicine injection device may control the driver to operate.

According to an embodiment, after the above-described step 640, the medicine cartridge may be removed from the medicine storage unit.

For example, while the tracked position of the piston is the initial position, the medicine cartridge may be removed from the medicine storage unit by the user.

The controller may detect a removal of the medicine cartridge form the medicine storage unit.

Upon detecting a removal of the medicine cartridge, the controller may set the locked driver to an operable or drivable state.

For example, the controller may release the advance-locked state of the driver.

However, without limitations thereto, the controller may release the locked state of the driver in response to a mounting of a new medicine cartridge in the medicine storage unit after the existing deteriorated medicine cartridge has been removed.

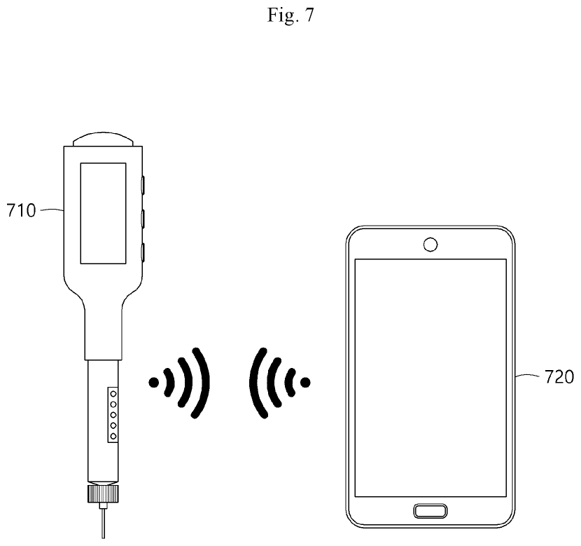

FIG. 7 is a view illustrating an example of communication with a user terminal according to an embodiment.

According to an embodiment, a medicine injection device 710 may further include a communication unit for communicating with a user terminal 720.

The user terminal 720 may be a terminal on which an application associated or interlocked with the medicine injection device 710 is installed and executed.

The user terminal 720 may be, e.g., a smartphone.

The medicine injection device 710 may establish wired or wireless communication with the user terminal 720 via the communication unit.

The medicine injection device 710 may transmit at least one of the medicine state and measured temperature to the user terminal 720 via the communication unit.

For example, the medicine injection device 710 may transmit data based on the temperature measured in the medicine injection device to the user terminal 720 via wireless communication.

The medicine injection device 710 may provide the user with the measurement result via an output unit (e.g., a display) of the user terminal 720 by transferring the result of measurement to the user terminal 720 via the communication unit.

The user terminal 720 may receive the measurement result from the medicine injection device 710 and store a measurement history associated with the results of measurement.

The measurement results may include the temperature measured in the medicine injection device 710 and the medicine state determined by the medicine injection device 710.

For example, as described above in connection with FIG. 4, the medicine injection device 710 may monitor the temperature associated with the medicine in the sleep mode, over time.

The medicine injection device 710 may record the medicine temperature predicted in each predetermined period.

The medicine injection device 710 may record the event (e.g., a danger event) in which the predicted medicine temperature exceeds the first threshold temperature and the event (e.g., a deterioration event) in which the predicted medicine temperature exceeds the second threshold temperature, along with the times of occurrence of the events.

When activated (e.g., waking up) from the sleep mode, the medicine injection device 710 may check the monitored medicine temperature history and the events caused during the sleep mode.

When at least one event of the danger event and the deterioration event occurs during the sleep mode, the medicine injection device 710 may transmit the time of occurrence of the event, along with the type of event (e.g., danger event or deterioration event), in response to an establishment of communication with the user terminal 720 after the medicine injection device 710 has been activated.

For example, the user terminal 720 may provide the user with a danger alarm in response to a reception of danger event-related information.

As another example, the user terminal 720 may provide the user with a deterioration alarm in response to a reception of deterioration event-related information.

Upon receiving the deterioration event, the user terminal 720 may transmit a signal to instruct to lock the driver to the medicine injection device 710.

Upon detecting a replacement of the cartridge or receiving a control or indication indicating the cartridge has been replaced from the user, the user terminal 720 may transmit a signal to instruct to release the locking of the driver (e.g., to unlock the driver) to the medicine injection device 710.

The user terminal 720 may receive the measured temperature via the communication unit of the medicine injection device 710, and the user terminal 720 may determine the medicine state based on the received temperature.

The communication unit of the medicine injection device 710 may receive the signal indicating the medicine state determined by the user terminal 720, and the medicine injection device 710 may control the driver based on the received signal.

Upon detecting the user's stop control input by the user terminal 720, the medicine injection device 710 may control the driver.

The above-described embodiments may be implemented as hardware components, software components, and/or in a combination thereof.

For example, the devices, methods, and components described herein may be implemented using one or more general-purpose or specific-purpose computers, such as processing devices, processors, controllers, arithmetic logic units (ALUs), digital signal processors, micro-computers, field programmable gate arrays (FPGAs), programmable logic units (PLUs), micro-controllers, any other devices capable of executing and responding to instructions.

The processing device or processor may perform an operating system (OS) and one or more software applications performed on the OS.

The processing device or processor may access, store, manipulate or control, process, and generate data in response to the execution of the software.

For illustration purposes, the processing device or processor may be a single one but it will be appreciated by one of ordinary skill in the art that a processing device may include a plurality of processing elements and/or a plurality of types of processing elements.

For example, the processing device may include a plurality of processors or a single processor and a single controller.

The processing device may have other various processing configurations, such as parallel processors.

The software may include computer programs, codes, instructions, or combinations of one or more thereof and may configure the processing device as it is operated as desired or may instruct the processing device independently or collectively.

The software and/or data may be embodied permanently or temporarily in signal waves transmitted or computer storage media or devices, virtual equipment, physical devices, components, or any type of machine so as to provide instructions or data to the processing device or to be interpreted by the processing device.

The software may be distributed over computer systems connected together via a network to be distributively stored or executed.

The software and data may be stored in one or more computer readable recording media.

The methods according to the embodiments of the present disclosure may be implemented in the form of programming commands executable by various computer means, and the programming commands may be recorded in a computer-readable medium.

The computer-readable medium may include programming commands, data files, or data structures, alone or in combinations thereof.

The programming commands recorded in the medium may be specially designed and configured for the present disclosure or may be known and available to one of ordinary skill in the computer software-related art.

Examples of the computer readable recording medium may include, but is not limited to, magnetic media, such as hard disks, floppy disks or magnetic tapes, optical media, such as CD-ROMs or DVDs, magneto-optical media, such as floptical disks, memories, such as ROMs, RAMs, or flash memories, or other hardware devices specially configured to retain and execute programming commands.

Examples of the programming commands may include, but are not limited to, high-level language codes executable by a computer using, e.g., an interpreter, as well as machine language codes as created by a compiler.

The above-described hardware devices may be configured to operate as one or more software modules to perform operations according to an embodiment of the present disclosure, or the software modules may be configured to operate as one or more hardware modules to perform the operations.

Although the disclosure is shown and described in connection with embodiments, it will be easily appreciated by one of ordinary skill in the art that various changes or modifications may be made without departing from the scope of the disclosure.

For example, although the techniques described herein are performed in a different order from those described herein and/or the components of the above-described structure or device are coupled, combined, or assembled in a different form from those described herein, or some components are replaced with other components or equivalents thereof, a proper result may be achieved.

What is claimed is:

1. A medicine injection device, comprising:
a medicine storage unit receiving a medicine cartridge storing a medicine;
a needle unit connected with the medicine cartridge and injecting the medicine to a user;
a driver configured to apply an external force to the medicine storage unit;
a temperature sensor measuring a temperature of at least one of the medicine storage unit, the needle unit, the driver, and an outside of the medicine injection device; and
a controller determining a medicine state indicating whether the medicine deteriorates based on the temperature measured by the temperature sensor and controlling the driver according to the determined medicine state to thereby determine whether to block the injection of the medicine.

2. The medicine injection device of claim 1, wherein when the temperature measured by the temperature sensor is less than a second threshold temperature and not less than a first threshold temperature, the controller determines that the medicine state is a danger state in which the medicine has a chance of deterioration.

3. The medicine injection device of claim 2, wherein when the temperature measured by the temperature sensor is not less than the second threshold temperature, the controller determines that the medicine state is a deteriorated state in which the medicine deteriorates.

4. The medicine injection device of claim 1, further comprising an alarm unit outputting an alarm indicating the medicine state determined by the controller, wherein when the controller determines that the medicine is in a danger state, the alarm unit outputs a danger alarm and, when the controller determines that the medicine is in a deteriorated state, the alarm unit outputs a deterioration alarm.

5. The medicine injection device of claim 1, wherein upon determining that the medicine state is a deteriorated state, the controller stops the operation of the driver to block the injection of the medicine.

6. The medicine injection device of claim 1, wherein in response to a controlling of the medicine injection by the controller, the driver moves a piston of the medicine cartridge received in the medicine storage unit from a rear end of the medicine injection device to a front end of the medicine injection device.

7. The medicine injection device of claim 6, wherein upon determining that medicine state is a deteriorated state, the controller controls the driver to block the injection of the medicine and, in response to a return, to an initial position, of the piston, allows the injection of the medicine.

8. The medicine injection device of claim 7, wherein after the medicine injection is blocked, the driver moves the piston from the front end of the medicine injection device to the initial position of the rear end.

9. The medicine injection device of claim 6, wherein upon determining that the medicine state is the deteriorated state, the controller locks an advance of the piston by the driver, tracks the position of the piston after the driver is locked, and in response to a determination that the tracked position is the initial position, sets the locked driver to an operable state.

10. The medicine injection device of claim 6, wherein in response to detecting a removal of the medicine cartridge from the medicine storage unit after the medicine injection is blocked, the controller sets the driver to an operable state.

11. The medicine injection device of claim 1, wherein when temperatures are measured in a plurality of positions of the medicine storage unit, the needle unit, the driver, and the outside of the medicine injection device, the controller determines the medicine state based on a highest temperature of the measured temperatures.

12. The medicine injection device of claim 1, wherein when temperatures are measured in a plurality of positions of the medicine storage unit, the needle unit, the driver, and the outside of the medicine injection device, the controller determines the medicine state based on an average temperature of the measured temperatures.

13. The medicine injection device of claim 1, further comprising a communication unit communicating with a user terminal, wherein a result of the measurement is transferred to the user terminal via the communication unit, and wherein a measurement history associated with the measurement result is stored in the user terminal.

14. The medicine injection device of claim 1, further comprising a communication unit communicating with a user terminal, wherein the communication unit receives a signal indicating the medicine state determined by the user terminal, and wherein the controller controls the driver based on the signal to thereby determine whether to block the injection of the medicine.

15. The medicine injection device of claim 14, wherein in at least one of a case where the medicine is determined to deteriorate by the user terminal or a case where the user's stop control input is detected, a stop control signal is received from the user terminal.

16. A method for operating a medicine injection device, the method comprising:
measuring a temperature of at least one of a medicine storage unit, needle unit, driver, and an outside of the medicine injection device;
determining a medicine state indicating whether the medicine deteriorates based on the measured temperature; and
controlling the driver according to the determined medicine state to thereby determine whether to block the medicine injection.
